(19) United States
(12) Patent Application Publication
(10) Pub. No.: US 2024/0173135 A1
(43) Pub. Date: May 30, 2024

(54) ARTIFICIAL VASCULAR VALVE

(71) Applicant: KONINKLIJKE PHILIPS N.V., EINDHOVEN (NL)

(72) Inventor: Andrew John PELTOMA, SAINT MICHAEL, MN (US)

(21) Appl. No.: 18/284,087

(22) PCT Filed: Mar. 24, 2022
(86) PCT No.: PCT/EP2022/057775

§ 371 Date: Sep. 26, 2023

Related U.S. Application Data
(60) Provisional application No. 63/168,359, filed on Mar. 31, 2021.

(51) Int. Cl. A61F 2/24; A61F 2/00; A61F 2/844

(52) U.S. Cl. CPC A61F 2/2475; A61F 2/0077; A61F 2/2406; A61F 2/844; A61F 2210/0014; A61F 2230/0006; A61F 2230/0065

(57) ABSTRACT

Vascular valves are disclosed. A vascular valve has a ring (101), and a valve flap (102) adapted to open and close based on vascular pressure. The valve flap (102) includes ribs (105) extending across at least a portion of the valve flap (102), and the ring (101) provides support to the valve flap (102) during and after deployment of the vascular valve.

### BACKGROUND

The deep venous system of the legs includes a series of valves preventing the back flow of blood as the calf pump and diaphragm pump blood back to the heart. Chronic deep vein thrombosis can damage these valves, preventing them from functioning properly, resulting in reflux.

To ensure proper blood flow, damaged valves can be replaced with artificial vascular valves. Notably, however, the valves in the legs are comparatively large (e.g., 6 mm in diameter). Known artificial vascular valves suitable for replacing damaged valves are thus often too large to be deployed using a catheter, at least because the venous path traversed includes veins that are too small to pass the known artificial vascular valve.

What is needed, therefore, is an artificial vascular valve for deployment in a comparatively large artery that overcomes at least the shortcomings of the known artificial vascular valves described above.

### SUMMARY

According to an aspect of the present disclosure, a vascular valve comprises: a ring; and a valve flap adapted to open and close based on vascular pressure, the valve flap comprising ribs extending across at least a portion of the valve flap, wherein the ring provides support to the valve flap during and after deployment of the vascular valve.

According to another aspect of the present disclosure, a vascular valve comprises: ring; a support element disposed in the ring and extending from one side of the ring to another side of the ring; and first and second valve flaps disposed on opposing sides of the ring, the first and second valve flaps being adapted to open and close based on vascular pressure, wherein the ring provides support to the first and second valve flaps during and after deployment.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A shows an artificial vascular valve in a closed position in accordance with a representative embodiment.

FIG. 1B shows the artificial vascular valve of FIG. 1A in an open position in accordance with a representative embodiment.

FIG. 1C shows a front view of the artificial vascular valve of FIG. 1A.

FIG. 1D shows a rear view of the artificial vascular valve of FIG. 1A.

FIG. 1E shows a side view of the artificial vascular valve of FIG. 1A.

FIG. 2A shows an artificial vascular valve in a closed position in accordance with a representative embodiment.

FIG. 2B shows the artificial vascular valve of FIG. 2A in an open position in accordance with a representative embodiment.

FIG. 2C shows a front view of the artificial vascular valve of FIG. 2A.

FIG. 2D shows a rear view of the artificial vascular valve of FIG. 2A.

FIG. 2E shows a side view of the artificial vascular valve of FIG. 2A.

FIG. 3A shows a front view and a side view of an artificial vascular valve in a folded artificial vascular valve state in accordance with a representative embodiment.

FIG. 3B shows a front view and a side view of an artificial vascular valve in a folded state ready for deployment in accordance with a representative embodiment.

FIG. 3C shows an artificial vascular valve disposed in a catheter adapted to be moved to a vein for deployment in accordance with a representative embodiment.

FIG. 3D shows the artificial vascular valve and the catheter of FIG. 3C after deployment into the vein in accordance with a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

As used in the specification and the appended claims and in addition to its ordinary meaning, the term ‘approximately’ means to within an acceptable limit or amount to one having ordinary skill in the art. For example, ‘approximately the same’ means that one of ordinary skill in the art would consider the items being compared to be the same.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

Relative terms, such as “above,” “below,” “top,” “bottom,” “upper”, “lower” “front” and “rear” may be used to describe the various elements' locations to one another, as illustrated in the accompanying drawings. These relative terms are intended to encompass different orientations of the device and/or elements in addition to the orientation depicted in the drawings. For example, if the device were rotated by 180° with respect to the view in the drawings, an element described as in the “front” of an element, for example, would now be to the “rear” of that element. Similarly, if the device were rotated by 90° with respect to the view in the drawings, an element described “above” or “below” another element would now be “adjacent” to the other element; where “adjacent” means either abutting the other element, or having one or more layers, materials, structures, etc., between the elements.

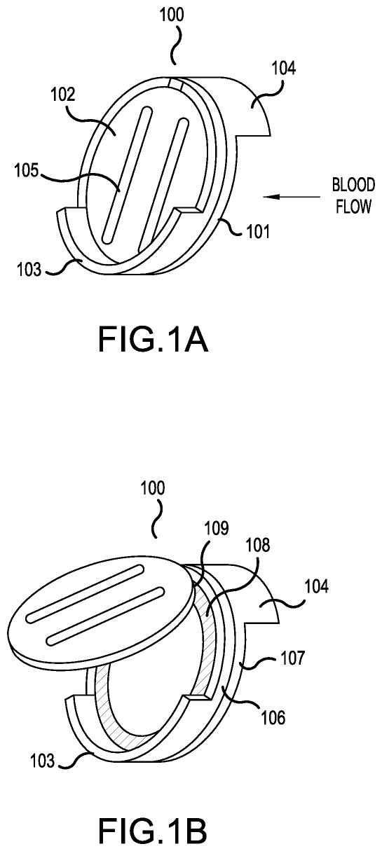

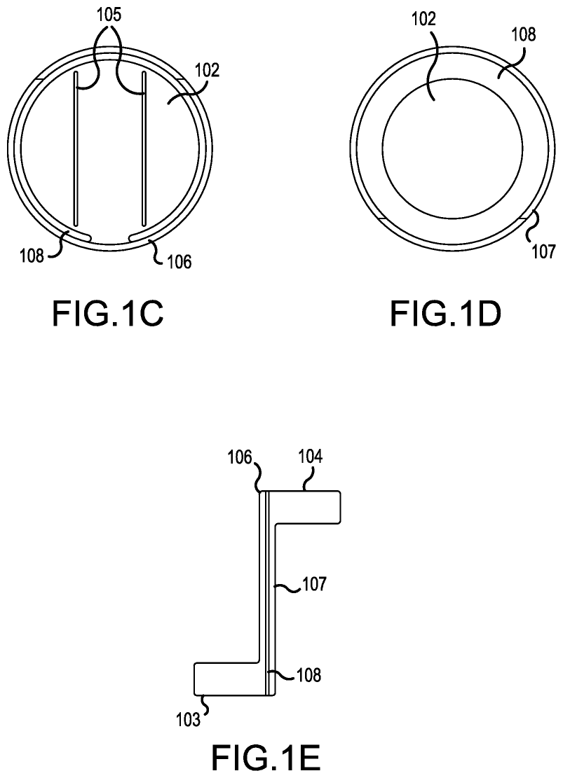

FIG. 1A shows an artificial vascular valve 100 in a closed position in accordance with a representative embodiment. The artificial vascular valve 100 comprises a ring 101, which comprises a front fin 103 and a rear fin 104. Notably, the ring 101 may comprise two separate rings: a front ring 106, which comprises the front fin 103; and a rear ring 107 which comprises the rear fin 104.

A valve flap 102 is disposed inside the ring 101 and comprises ribs 105 that extend at least partially along from one side of the valve flap 102 to the other. In its state shown in FIG. 1A, the valve flap 102 is in a closed position to prevent blood from flowing back from the artificial vascular valve 100. As described more fully below, the valve flap 102 is rotated upward about a pivot point to open the artificial vascular valve to allow blood pumped from the heart to flow.

For reasons that will become more clear as the present description continues, the ring 101 (as a single ring or comprising front and rear rings 106, 107) are made of a memory metal, such as nickel-titanium alloy commonly known as nitinol. The state of the ring 101 (or front and rear rings 106, 107) shown in FIG. 1A is referred to herein as an original state when the artificial vascular valve 100 is deployed. To effect deployment of the artificial vascular valve 100, the ring 101 (or front and rear rings 106, 107) are folded to have a small diameter to foster deployment via a catheter as described more fully below.

The valve flap 102 and the ribs 105 may be made of a material suitable to artificial vascular valves, such as a suitable molded polymer. In a representative embodiment, the valve flap 102 and ribs may be made of graphite coated with pyrolytic carbon. Notably, the ribs 105 provide a degree of stiffness to the structure.

FIG. 1B shows the artificial vascular valve 100 of FIG. 1A in an open position in accordance with a representative embodiment. In the open position, a valve flap support 108 is disposed along the inside of the rear ring 107 to provide a location for fastening the valve flap 102 to the rear ring 107, and to provide further support.

When blood is pumped by the heart to flow in the direction shown, the valve flap 102 is rotated by a pivot 109 to allow the blood to flow through the artificial vascular valve 100. As will be appreciated, pressure during the contraction of the heart causes the valve flap 102 to open. When the heart expands after a contraction, the pressure then changes and the valve flap 102 pivots to the position shown in FIG. 1A. In this state, the artificial vascular valve 100 is closed and prevents blood from flowing improperly.

Fabrication of the artificial vascular valve 100 comprises cutting the front and rear fins 103, 104, which are then laser welded to the front and rear rings 106, 107, respectively. Thereafter, the front and rear fins 103, 104 and front and rear rings 106, 107 are heat set and electropolished using known techniques so the original/deployed state of the front and rear fins 103, 104 and front and rear rings 106, 107 (and thus artificial vascular valve 100) are fixed.

The valve flap support 108 and the valve flap 102, comprising ribs 105 are cut from bulk material. The valve flap support 108 and valve flap 102 are sandwiched and adhered between two front and rear rings 106, 107.

FIG. 1C shows a front view of the artificial vascular valve 100 of FIG. 1A. Specifically, the front view of the artificial vascular valve 100 of FIG. 1A depicted shows the artificial vascular valve 100 in a closed position with the front ring 106 and the valve flap support 108 visible. Notably, the front fin 103 is not clearly shown in FIG. 1C, and a portion of the valve flap support 108 is partially covered by the front ring 106. In the state shown, the front ring 106 is in the original/deployed state. As will become clearer as the present description continues, the front and rear rings 106, 107 are folded for deployment through a catheter, and after deployment by the catheter return to the original state.

FIG. 1D shows a rear view of the artificial vascular valve 100 of FIG. 1A. Specifically, the rear view of the artificial vascular valve 100 of FIG. 1A depicted shows the artificial vascular valve 100 in a closed position with the rear ring 107 and the valve flap support 108 visible. Notably, the rear fin 104 is not clearly shown in FIG. 1D. In the state shown, the rear ring 107 is in the original/deployed state. As will become clearer as the present description continues, the front and rear rings 106, 107 are folded for deployment through a catheter, and after deployment by the catheter return to the original state.

FIG. 1E shows a side view of the artificial vascular valve 100. Specifically, the side view of the artificial vascular valve 100 of FIG. 1A depicted shows the front fin 103; the front ring 106; the rear fin 104; the rear ring 107; and the valve flap support 108 disposed between the front and rear rings 106, 107. Again, in the state shown, the front and rear rings 106.107 are in the original/deployed state. As will become clearer as the present description continues, the front and rear rings 106, 107 are folded for deployment through a catheter, and after deployment by the catheter return to the original state.

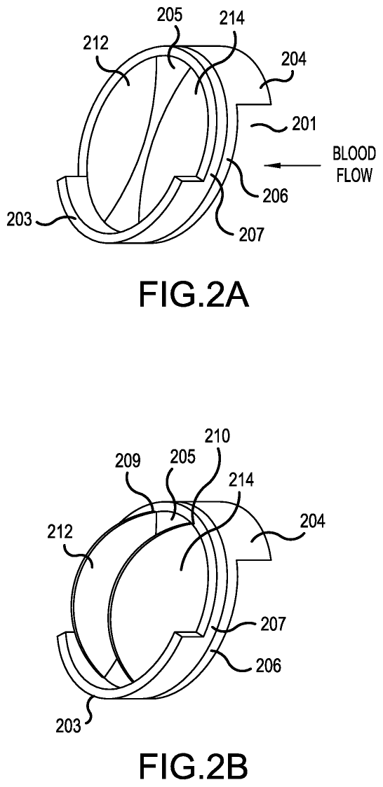

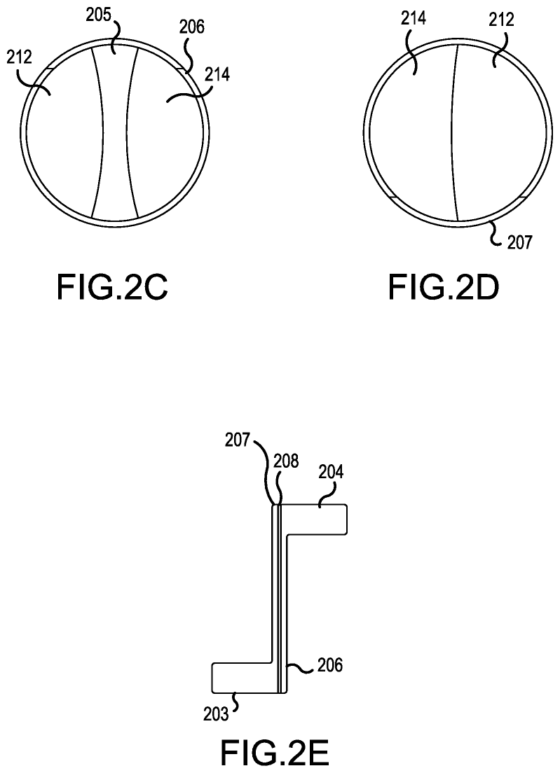

FIG. 2A shows an artificial vascular valve 200 in a closed position in accordance with a representative embodiment. Many aspects and details of the artificial vascular valve 200 are common to those of the artificial vascular valve 100 discussed above in connection with the representative embodiments of FIGS. 1A-1E. These common aspects and details may not be repeated to avoid obscuring the presently described representative embodiments.

The artificial vascular valve 200 comprises a ring 201, which comprises a front fin 203 and a rear fin 204. Notably, the ring 201 may comprise two separate rings: a front ring 206, which comprises the front fin 203; and a rear ring 207 which comprises the rear fin 204.

A first valve flap 212 and a second valve flap 214 are disposed inside the ring 201. A support element 205 is disposed between opposing sides of the ring 201. In its state shown in FIG. 2A, the first and second valve flaps 212, 214 are in a closed position to prevent blood from flowing back from the artificial vascular valve 200. As described more fully below, the first and second valve flaps 212, 214 each rotate about first and second pivot points to open the artificial vascular valve to allow blood pumped from the heart to flow.

For reasons that will become clearer as the present description continues, the ring 201 (as a single ring or comprising front and rear rings 206, 207) and the support element 205 are made of a memory metal, such as nickel-titanium alloy commonly known as nitinol. The state of the ring 201 (or front and rear rings 206, 207) shown in FIG. 2A is referred to herein as an original state when the artificial vascular valve 200 is deployed. To effect deployment of the artificial vascular valve 200, the ring 201 (or front and rear rings 206, 207) are folded to have a small diameter to foster deployment via a catheter as described more fully below.

The first and second valve flaps 212, 214 may be made of a material suitable to artificial vascular valves, such as graphite coated with pyrolytic carbon.

FIG. 2B shows the artificial vascular valve 200 of FIG. 2A in an open position in accordance with a representative embodiment.

When blood is pumped by the heart to flow in the direction shown, the first and second valve flaps 212, 214 are rotated at first and second pivots 209, 210, respectively, to allow the blood to flow through the artificial vascular valve 200. When blood is pumped by the heart to flow in the direction shown, the valve flap 102 is rotated by a pivot 109 to allow the blood to flow through the artificial vascular valve 100. As will be appreciated, pressure during the contraction of the heart causes the first and second valve flaps 212, 214 to open. When the heart expands after a contraction, the pressure then changes and the first and second valve flaps 212, 214 are rotated to the position shown in FIG. 2A. In this state, the artificial vascular valve 200 is closed to prevent blood from flowing improperly.

Fabrication of the artificial vascular valve 200 comprises cutting the front and rear fins 203, 204, which are laser welded to the front and rear rings 206, 207, respectively. Thereafter, the front and rear fins 203, 204, the support element 205, and the front and rear rings 206, 207 are heat set and electropolished using known techniques so the original/deployed state of the front and rear fins 203, 204, the support element 205, and the front and rear rings 206, 207 (and thus artificial vascular valve 200) are fixed.

FIG. 2C shows a front view of the artificial vascular valve 200 of FIG. 2A. Specifically, the front view of the artificial vascular valve 200 of FIG. 1A depicted shows the artificial vascular valve 200 in a closed position with the front ring 206, the first and second valve flaps 212, 214 and the support element 205 visible. Notably, the front fin 203 is not clearly shown in FIG. 2C. In the state shown, the front ring 206 and the support element 205 are in the original/deployed state. As will become clearer as the present description continues, the support element 205 and the front and rear rings 206, 207 are folded for deployment through a catheter, and after deployment by the catheter return to the original state.

FIG. 2D shows a rear view of the artificial vascular valve 200 of FIG. 1A. Specifically, the rear view of the artificial vascular valve 200 of FIG. 2A depicted shows the artificial vascular valve 200 in a closed position with the rear ring 207 shown. Notably, the rear fin 204 is not clearly shown in FIG. 2D. In the state shown, the rear ring 207 is in the original/deployed state. As will become clearer as the present description continues, the support element 205, and the front and rear rings 206, 207 are folded for deployment through a catheter, and after deployment by the catheter return to the original state.

FIG. 2E shows a side view of the artificial vascular valve 200. Specifically, the side view of the artificial vascular valve 200 of FIG. 1A depicted shows the front fin 203; the front ring 206; the rear fin 204; the rear ring 207; and a valve flap support 208 disposed between the front and rear rings 206, 207. Again, in the state shown, the front ring and rear rings 206, 207 is in the original/deployed state. As will become clearer as the present description continues, the support element 205 and the front and rear rings 206, 207 are folded for deployment through a catheter, and after deployment by the catheter return to the original state.

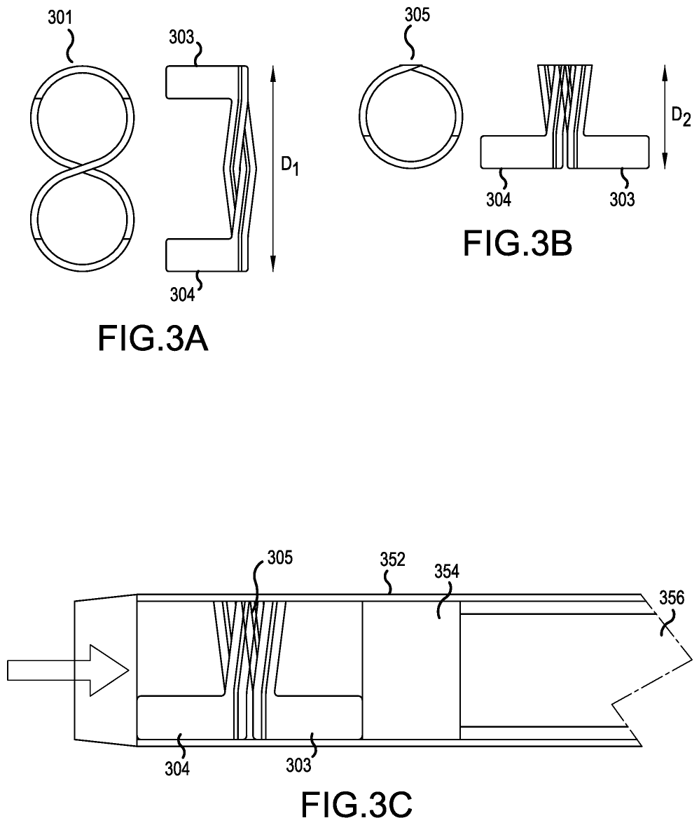

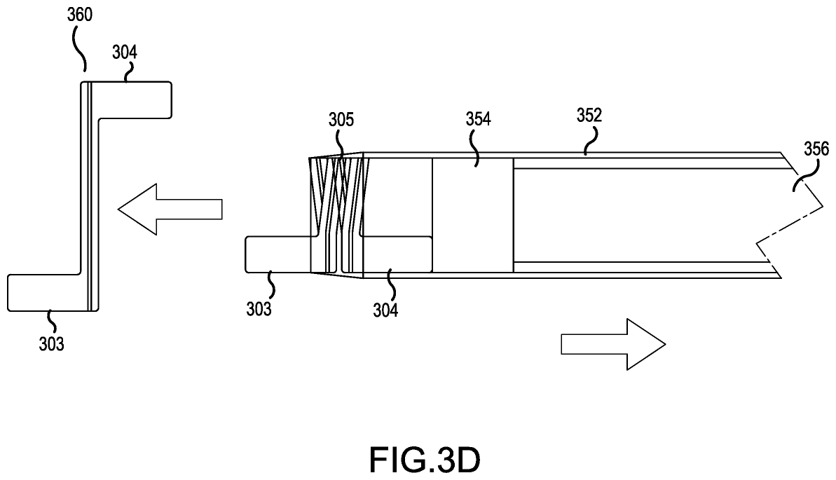

FIG. 3A shows a front view and a side view of an artificial vascular valve 300 in a folded artificial vascular valve state in accordance with a representative embodiment. Many aspects and details of the artificial vascular valve 300 are common to those of the artificial vascular valve 100 discussed above in connection with the representative embodiments of FIGS. 1A-2E. These common aspects and details may not be repeated to avoid obscuring the presently described representative embodiments.

The artificial vascular valve 300 in FIG. 3A is in the shape of a “FIG. 8,” which is done by folding artificial vascular valve 100 or artificial vascular valve 200 from an initial circular state (e.g., in FIG. 1A or FIG. 2A). This fold causes the artificial vascular valve 300 to form first and second circles 306, 308 as shown. As alluded to above, the folding of the artificial vascular valves 100, 200 is done to reduce the size of the artificial vascular valve to be transferred via a catheter. After being properly located, the catheter is withdrawn, and the artificial vascular valve, because of its components' being made of a memory material such as described above, returns to its initial state having a comparatively large dimension to function in a comparatively large vein.

As shown in FIG. 3A, the FIG. 8 shape of artificial vascular valve 300 has a length D1 that is equal to the diameter of artificial vascular valve 100 or artificial vascular valve 200 in an initial circular state (e.g., in FIG. 1A or FIG. 2A). In this state, front and rear fins 303, 304 are as shown.

FIG. 3B shows a front view and a side view of an artificial vascular valve 305 in another folded state ready for deployment in accordance with a representative embodiment. Specifically, the artificial vascular valve 300 in FIG. 3A is folded a second time so one of the first and second circles 306, 308 is disposed over the other of the first and second circles 306, 308. In this position, the artificial vascular valve 305 has a length D2, which is substantially equal to a diameter of the first and second circles 306, 308. When folded into the state shown in FIG. 3B, the artificial vascular valve 305 is small enough to be fit into a catheter for deployment. After being properly located, the catheter is withdrawn, and the artificial vascular valve 305, because of its components' being made of a memory material such as described above, returns to its initial state having a comparatively large dimension to function in a comparatively large vein.

FIG. 3C shows artificial vascular valve 305 disposed in a catheter 352 adapted to be moved to a vein for deployment in accordance with a representative embodiment. Many aspects and details of the artificial vascular valve 300 are common to those of the artificial vascular valve 100 discussed above in connection with the representative embodiments of FIGS. 1A-3B. These common aspects and details may not be repeated to avoid obscuring the presently described representative embodiments.

Notably, the artificial vascular valve 305 in its comparatively small state is loaded (shown by arrow) into one end of the catheter. The catheter 352 which comprises a sheath, is shown with a pusher 354 and a pusher shaft 356 in an opposing end. In this state, the catheter can be fed into a vein for deployment of the artificial valve.

FIG. 3D shows the artificial vascular valve 305 the catheter of FIG. 3C after deployment into the vein in accordance with a representative embodiment. Specifically, after the catheter 352 is properly located in the vein, the pusher shaft 356 is moved (e.g., manually) and the pusher moves the artificial vascular valve 305 (as shown by the arrow) out of the catheter 352. As shown, the artificial vascular valve 360 with front and rear fins 303, 304 positioned as shown. As will be appreciated, the various components of the artificial vascular valve 305 made of memory material as noted above, upon exiting the catheter 352 return to their original state, and the artificial vascular valve 360 is in a deployed state. As noted above, in its deployed state, the artificial vascular valve 360 may be one of artificial vascular valve 100 or artificial vascular valve 200 shown and described above in connection with FIGS. 1A-2E.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Although an artificial vascular valve, and techniques and components for its deployment have been described with reference to several representative embodiments, it is understood that the words that have been used are words of description and illustration, rather than words of limitation. Changes may be made within the purview of the appended claims, as presently stated and as amended, without departing from the scope and spirit of artificial vascular valves of the present teachings in its aspects. Although the artificial vascular valves and their deployment have been described with reference to particular means, materials and embodiments, the artificial vascular valves are not intended to be limited to the particulars disclosed; rather the artificial vascular valves extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable a person ordinarily skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A vascular valve, comprising:
a ring; and
a valve flap adapted to open and close based on vascular pressure, the valve flap comprising ribs extending across at least a portion of the valve flap, wherein the ring provides support to the valve flap during and after deployment of the vascular valve.

2. The vascular valve of claim 1, wherein the ring is a front ring and the vascular valve further comprises a read ring, and the front and rear ring each have a substantially circular shape.

3. The vascular valve of claim 2, wherein the front and rear rings are adapted to fold during deployment of the vascular valve, and to unfold to an original shape when deployment of the vascular valve is completed.

4. The vascular valve of claim 2, wherein the front ring has a substantially circular shape, and the ribs have a length that is less than a diameter of the front ring.

5. The vascular valve of claim 4, further comprising a first fin disposed at a lower surface of the front ring, and a second fin disposed at an upper surface of the rear ring.

6. The vascular valve of claim 2, wherein the front and rear rings ring each comprise a memory material.

7. The vascular valve of claim 6, wherein the memory material comprises a nickel-titanium alloy.

8. The vascular valve of claim 2, further comprising a pivot along one portion of the front ring.

9. A vascular valve, comprising:
a ring;
a support element disposed in the ring and extending from one side of the ring to another side of the ring; and
first and second valve flaps disposed on opposing sides of the ring, the first and second valve flaps being adapted to open and close based on vascular pressure, wherein the ring provides support to the first and second valve flaps during and after deployment.

10. The vascular valve of claim 9, wherein the ring is a front ring, and the vascular valve further comprises a rear ring, the front and rear rings being adapted to fold during deployment of the vascular valve, and to unfold to an original shape when deployment of the vascular valve is completed.

11. The vascular valve of claim 10, wherein the rear ring and the front ring are each have a substantially circular shape.

12. The vascular valve of claim 10, wherein the front ring has a substantially circular shape, and the support element extends across a diameter of the front ring and between the first and second valve flaps.

13. The vascular valve of claim 12, further comprising a first fin disposed at a lower surface of the front ring and a second fin disposed at an upper surface of the rear ring.

14. The vascular valve of claim 10, wherein the rear ring and the front ring each comprise a memory material.

15. The vascular valve of claim 14, wherein the memory material comprises a nickel-titanium alloy.

16. The vascular valve of claim 10, further comprising a first pivot along one portion of the front ring, and a second pivot along an opposing portion of the front ring.
